(19) United States
(12) Patent Application Publication
(10) Pub. No.: US 2011/0275128 A1
(43) Pub. Date: Nov. 10, 2011

(54) Use Of Whey For The Manufacture Of Erythritol

(75) Inventor: Thomas Lee, Scarsdale, NY (US)

(73) Assignee: PepsiCo, Inc., Purchase, NY (US)

(21) Appl. No.: 13/095,960

(22) Filed: Apr. 28, 2011

Related U.S. Application Data
(60) Provisional application No. 61/331,424, filed on May 5, 2010.

(51) Int. Cl. C12P 7/26; C12P 7/18

(52) U.S. Cl. 435/148; 435/158

(57) ABSTRACT

Erythritol is prepared from whey. Whey is treated to form lactose, the lactose is treated to form glucose and galactose, the glucose and galactose are separated, and then the glucose is treated to form erythritol.

This application claims priority to U.S. Provisional Application No. 61/331,424, filed May 5, 2010, and hereby incorporated by reference in its entirety.

### FIELD OF THE INVENTION

The invention relates to a process of making erythritol using whey as a starting material.

### BACKGROUND OF THE INVENTION

Erythritol and tagatose have commercial utility as low and no calorie bulking agents, as taste modifiers, and in beverages such as frozen carbonated beverages, see for example, U.S. Pat. No. 7,579,032. However, the supply of tagatose and erythritol has been limited due to, for example, high cost of the raw material and the processes of making these ingredients.

Erythritol ((2R,3S)-butane-1,2,3,4-tetraol) is a natural sugar alcohol which has been approved for use through the world including the United States. Eryrthritol occurs naturally in fruits and fermented foods. In industry, it is produced from glucose by fermentation with a yeast or fungi such as Mondiella pollinis. Suitable yeast or fungi can be found in Shie-Jea Lin, et.al. “Screening and production of erythritol by newly isolated osmophilic yeast-like fungi, in Process Biochemistry, vol. 36, issue 12 (2001.) It is about 65% as sweet as table sugar but is almost non-caloric, does not affect blood sugar, does not cause tooth decay, and is absorbed by the body. Unlike other sugar alcohols, it is unlikely to cause gastric side effects.

Erythritol is normally made from glucose that is created from corn or wheat starch. The starch is first treated with enzymes (special proteins) that break the starch down into glucose. This glucose is then mixed with yeast, such as Mondiella pollinis or Trichosporonoides megachliensis, and the yeast ferments the glucose to form erythritol. The fermented mixture is then heated (in order to kill off the yeast) and dried (by boiling off all the water) so that erythritol crystals are formed. These crystals are then washed (to remove impurities), redissolved, purified again (using a special kind of chemical filter) and finally are isolated in solid form, at which point the erythritol is safe for human consumption.

There is great competition for sources of corn and wheat for food and ethanol production, for example, thus production of erythritol from these sources has become expensive. In addition, there is a significant amount of processing to form erythritol from corn, for example. The corn must be crushed, the husk removed, and ground into flour. Protein is removed to provide the corn starch which is a polymer of glucose. The starch is hydrolyzed to form glucose monomers in the form of corn syrup or glucose syrup. This syrup is then treated to form erythritol.

It is desired to use an alternative source for erythritol production. However, sources such as whey have not previously been considered, perhaps as the glucose is derived from an animal source.

### BRIEF SUMMARY OF THE INVENTION

One aspect of the invention is directed to a method for making erythritol comprising treating whey to form lactose, treating the lactose to form glucose and galactose, separating the glucose and galactose, and then treating the glucose to form erythritol.

Another aspect of the invention is directed to a method for making erythritol and tagatose comprising treating whey to form lactose, treating the lactose to form glucose and galactose, separating the glucose and galactose, then treating the galactose to form tagatose and treating the glucose to form erythritol.

Another aspect of the present invention is the treatment of waste glucose obtained from the production of tagatose from whey.

### DETAILED DESCRIPTION OF THE INVENTION

The present invention is directed to making erythritol from a whey starting material. Whey is a waste material from making cheese. Specifically whey is the liquid remaining after milk has been curdled and strained. Whey may be discarded. Alternatively, because whey generally contains greater than about 4.5% of lactose, it may be used to prepare products such as tagatose.

The milk used to prepare the whey is typically cow's milk or goat's milk. Milk is curdled to form cheese. The water portion and washing is called whey. Whey contains 1. lactose (about 4.5 to 5% w/v), 2. proteins (about 0.8-1.0% w/v, mainly whey proteins) and 3. minerals (about 0.5 to 0.7% w/v). Whey from cheese making is concentrated via evaporation. Using methods like ultrafiltration, nanofiltration and reverse osmosis, etc., one can isolate proteins for sale as “whey protein concentrate” or isolated whey protein. The permeate contains mostly lactose which can be separated by crystallization via cooling.

To make tagatose, the lactose is treated with an enzyme to hydrolyze into galactose and glucose, typically in a 1:1 ratio. The glucose is removed as waste, such as by chromatography such as passing the galactose/glucose solution through an immobilized lactase column, and then the galactose is treated to form tagatose via alkaline isomerization by adding, for example, Ca(OH)₂ and optionally a catalyst such as CaCl₂. The reaction is stopped by adding a technical-grade sulfuric acid.

It was discovered that the waste glucose provides a cheap source for producing erythritol. Thus, the present invention is directed to treating the waste glucose obtained from whey used in tagatose production to make erythritol. The glucose portion can be treated with the yeasts, fungi, or enzymes to form erythritol by cleaving two carbons away from glucose.

Suitable yeast or fungi can be found in Shie-Jea Lin, et.al. “Screening and production of E by newly isolated osmophilic yeast-like fungi, in Process Biochemistry, vol. 36, issue 12 (2001) Non-limiting examples are Mondiella pollinis and Trichosporonoides megachliensis. The mixture undergoes fermentation and the glucose is broken down into erythritol. The fermented mixture is then heated to kill off the yeast or deactivate the enzyme. After filtration, the solution is dried, for example by boiling off the water, so that erythritol crystals are formed.

These crystals may be treated to remove impurities. For example, the crystals are washed, redissolved, recrystallized, washed again, and then isolated in solid form. The erythritol is typically purified to at least 99.5% pure erythritol and is safe for human consumption.

Producing erythritol from the waste glucose stream significantly reduces the amount of waste from the production of tagatose. Thus the process is green. Another advantage is a low cost source of glucose for preparing the erythritol. A further advantage is that the overall cost of producing the tagatose is decreased as profit may be gleaned from the preparation of erythritol.

Another benefit is that whey is not a genetically modified (GMO) food. Corn grown in the United States is considered GMO-corn. Thus, non-GMO erythritol cannot be made using US corn. However, as milk (the starting product to make cheese and whey) is non-GMO, the erythritol made from whey will be non-GMO. This is important for countries in Europe where non-GMO erythritol is preferred.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

We claim:

1. A method for making erythritol comprising treating whey to form lactose, treating the lactose to form glucose and galactose, separating the glucose and galactose, then treating the glucose to form erythritol.

2. The method of claim 1 wherein the glucose and galactose are separated by chromatography.

3. The method of claim 1 wherein the glucose is treated with a yeast, fungi, or enzyme effective to form erythritol.

4. The method of claim 3 wherein the glucose is treated with Moniliella pollinis or Trichosporonoides megachliensis and fermented until the glucose is broken down into erythritol.

5. The method of claim 1 wherein the erythritol is crystallized.

6. The method of claim 5 wherein the crystallized erythritol is purified to at least 99.5% pure erythritol.

7. A method for making tagatose and erythritol comprising treating whey to form lactose, treating the lactose to form glucose and galactose, separating the glucose and galactose, then treating the glucose to form erythritol and treating the galactose to form tagatose.

8. The method of claim 7 wherein the glucose and galactose are separated by chromatography.

9. The method of claim 8 wherein the glucose is treated with a yeast or enzyme effective to form erythritol.

10. The method of claim 9 wherein the glucose is treated with Moniliella pollinis or Trichosporonoides megachliensis and fermented until the glucose is broken down into erythritol.

11. The method of claim 8 wherein the erythritol is crystallized.

12. The method of claim 13 wherein the crystallized erythritol is purified to at least 99.5% pure erythritol.

13. A method of treating waste glucose obtained from the production of tagatose from whey comprising treating whey to form lactose, treating the lactose to form glucose and galactose, separating the glucose and galactose, then treating the glucose to form erythritol.
